(19) United States
(12) Patent Application Publication
(10) Pub. No.: US 2025/0255919 A1
(43) Pub. Date: Aug. 14, 2025

(54) USE OF AQUEOUS EXTRACT FROM LINDERAE RADIX FOR PREPARATION OF MEDICAMENT FOR PREVENTING AND TREATING ULCERATIVE COLITIS

(71) Applicant: Zhejiang Chinese Medical University, Hangzhou (CN)

(72) Inventors: Zhaohuan LOU, Hangzhou (CN); Yingying WANG, Hangzhou (CN); Xunjie ZHENG, Hangzhou (CN); Jianbo HUANG, Hangzhou (CN); Ke LI, Hangzhou (CN); Weizhi LAI, Hangzhou (CN)

(21) Appl. No.: 19/170,007

(22) Filed: Apr. 3, 2025

(30) Foreign Application Priority Data
Apr. 7, 2024 (CN) 202410405601.4

(51) Int. Cl. A61K 36/54; A61P 1/04

(52) U.S. Cl. CPC A61K 36/54; A61P 1/04; A61K 2236/331; A61K 2236/39

(57) ABSTRACT

The use of an aqueous extract from Linderae Radix for the preparation of a medicament for preventing and treating ulcerative colitis is provided, which belongs to the technical field of prevention and treatment of ulcerative colitis. The present disclosure proposes for the first time that an aqueous extract from Linderae Radix has the effects of preventing and treating ulcerative colitis, wherein the aqueous extract from Linderae Radix can effectively alleviate the symptoms of ulcerative colitis, mainly including improving the clinical symptoms and pathological changes in mice with dextran sodium sulfate-induced ulcerative colitis. In addition, the present disclosure proposes for the first time that the aqueous extract from Linderae Radix has the effects of: maintaining the number and function of goblet cells; increasing the number of mitochondria; activating “bile acid-mitochondrial” signaling to enhance Lgr5⁺ ISC activity to further promote the regeneration of intestinal epithelial cells.

### CROSS REFERENCE TO RELATED APPLICATION

This patent application claims the benefit and priority of Chinese Patent Application No. 202410405601.4, entitled “USE OF AQUEOUS EXTRACT FROM LINDERAE RADIX FOR PREPARATION OF MEDICAMENT FOR PREVENTING AND TREATING ULCERATIVE COLITIS” filed with the China National Intellectual Property Administration on Apr. 7, 2024, the disclosure of which is incorporated by reference herein in its entirety as part of the present application.

### TECHNICAL FIELD

The present disclosure belongs to the technical field of prevention and treatment of ulcerative colitis, in particular relates to the use of an aqueous extract from Linderae Radix for the preparation of a medicament for preventing and treating ulcerative colitis.

### BACKGROUND

Ulcerative colitis (UC) is a chronic recurrent disease that mainly involves the colorectal mucosa and submucosa, with increasing incidence and prevalence year by year. The common clinical symptoms of UC are abdominal pain, diarrhea, bloody mucous stool, etc. The pathogenesis of UC is prolonged and repeated, which is an independent risk factor for colon cancer. At present, aminosalicylic acid, glucocorticoids, immunoregulatory drugs and biological preparations are mainly used clinically to treat UC, and these drugs can effectively reduce local inflammatory responses in the intestinal tract. However, only about 40% of patients achieve clinical remission, with the relapse rate as high as 54.4% to 79.2%. Studies have shown that incomplete intestinal epithelial barrier mediated by the death of intestinal epithelial cells (IECs) and subsequent inflammatory responses are the main causes of the onset and progression of UC. The failure to regenerate and repair intestinal epithelium in time after intestinal tract damage is the key to the non-healing of UC after anti-inflammatory treatment, and promoting the healing of intestinal epithelium is the ultimate goal of UC treatment. Therefore, it is of great significance to deeply reveal the mechanism of colonic epithelial regeneration of UC and to explore new strategies and new drugs for preventing and treating UC.

Linderae Radix is the dried root of Lindera aggregate (Sims) Kosterm. of the family Lauraceae, and has the functions of activating qi-flowing, dispersing stagnated liver qi, treating diarrhea and dysentery, etc. Extracts extracted from Linderae Radix by using different extraction solvents have different biological activities and pharmaceutical effects. New studies have found that Lgr5⁺ intestinal stem cells (ISCs) located at the base of colonic crypt replenish damaged IEC by means of rapid renewal differentiation to promote UC ulcer healing, and therefore, maintaining the number and activity of Lgr5⁺ ISC is necessary for regeneration and repair of intestinal epithelium after intestinal tract damage. At present, modulatory factors that affect Lgr5 expression and Lgr5⁺ ISC activity under inflammatory damage, particularly parenteral factors such as bile acids, are of great interest. Studies have found that pathological concentration of cholic acid (CA) directly inhibits the proliferation of Lgr5⁺ ISC and weaken the renewal ability of Lgr5⁺ ISC by inhibiting fatty acid beta-oxidation (FAO) to exacerbate damage to the intestinal epithelial barrier, secondary bile acids such as lithocholic acid (LCA) and Takeda G-protein-coupled receptor 5 [TGR5, also known as GPBAR1 (G-protein-coupled bile acid receptor 1)] thereof promote the self-renewal and differentiation of ISC and support the reconstitution of Lgr5⁺ ISC and intestinal epithelial regeneration after intestinal tract damage by activating Src/Yap and downstream target genes, and TGR5-Src/Yap is a key signaling pathway for bile acids to regulate ISC activity. In addition, mitochondrial function is closely associated with the self-renewal and differentiation potential of ISCs, and inflammation can damage the function of intestinal epithelial mitochondria, thereby reducing Lgr5 expression and affecting ISC activity. Mitochondrial function and ISC activity are regulated by mitochondrial dynamics. Insufficient mitochondrial fusion affects mitochondrial function to decrease ISC activity, thereby affecting the proliferation and differentiation of intestinal epithelial cells and mediating the occurrence and progression of UC. Therefore, enhancing intestinal stem cell activity by regulating bile acid-mitochondrial signaling is expected to promote intestinal epithelial regeneration, which provides a new strategy for breaking the bottleneck of non-healing after treatment of UC. At present, there are no relevant studies in the prior art that aqueous extracts from Linderae Radix have a regulatory effect on intestinal stem cells with ulcerative colitis and that aqueous extracts from Linderae Radix play an anti-UC role by activating “bile acids-mitochondrial” signaling to enhance Lgr5⁺ ISC activity to further promote the regeneration and repair of intestinal epithelial cells.

### SUMMARY

In view of this, an objective of the present disclosure is to provide the use of an aqueous extract from Linderae Radix in the preparation of a medicament for preventing and treating ulcerative colitis.

To achieve the above objective of the present disclosure, the present disclosure provides the following technical solutions:

The present disclosure provides the use of an aqueous extract from Linderae Radix in the preparation of a medicament for preventing and treating ulcerative colitis.

In some embodiments, a method for preparing the aqueous extract from Linderae Radix includes the steps of: adding Linderae Radix in water and soaking. reflux extracting the Linderae Radix with water for 1.0 h-2.0 h at 60° C.-80° C., recovering obtained crude extract under reduced pressure, and concentrating the crude extract to obtain the aqueous extract from Linderae Radix.

In some embodiments, the mass-to-volume ratio of the Linderae Radix to the water is 1 g:10 mL.

In some embodiments, a time required for the soaking is 30 min-60 min.

In some embodiments, the reflux extracting is performed for twice.

In some embodiments, the aqueous extract from Linderae Radix plays a role by maintaining the function of goblet cells and restoring intestinal mucus barrier.

In some embodiments, the aqueous extract from Linderae Radix plays a role by alleviating the damage to colonic epithelial mitochondria and increasing the number of mitochondria.

In some embodiments, the aqueous extract from Linderae Radix plays an anti-ulcerative colitis role by activating “bile acids-mitochondrial” signaling to enhance Lgr5⁺ ISC activity to further promote the regeneration and repair of intestinal epithelial cells.

In some embodiments, the ulcerative colitis is a dextran sodium sulfate-induced ulcerative colitis.

The present disclosure also provides a medicament for preventing and treating ulcerative colitis, where the medicament contains the aqueous extract from Linderae Radix prepared above as the sole active ingredient.

The present disclosure has the following beneficial effects:

The present disclosure proposes for the first time that an aqueous extract from Linderae Radix has the effects of preventing and treating ulcerative colitis, where the aqueous extract from Linderae Radix may effectively alleviate the symptoms of ulcerative colitis, mainly including improving the clinical symptoms and pathological changes in mice with dextran sodium sulfate-induced ulcerative colitis. In addition, the present disclosure proposes for the first time that the aqueous extract from Linderae Radix has the effects of: maintaining the function of goblet cells and restoring intestinal mucus barrier; alleviating the damage to colonic epithelial mitochondria and increasing the number of mitochondria; and playing an anti-ulcerative colitis role by activating “bile acid-mitochondrial” signaling to enhance Lgr5⁺ ISC activity to further promote the regeneration and repair of intestinal epithelial cells.

### BRIEF DESCRIPTION OF THE DRAWINGS

FIGS. 1A-1F show the prevention and treatment effects of the aqueous extract from Linderae Radix on DSS-induced ulcerative colitis (UC), where FIG. 1A: general observation of colon; FIG. 1B: colon length; FIG. 1C: change in mouse body weight; FIG. 1D: score of disease activity index (DAI); FIG. 1E: HE staining of colonic tissue (100×); and FIG. 1F: AB-PAS staining of colonic tissue (100×); when compared with normal control group (Normal), ***P<0.001; and when compared with model group, #P<0.05, ##P<0.01;

FIGS. 2A-2D show the effect of the aqueous extract from Linderae Radix (WY) on genes involved in bile acids metabolism regulation in UC mice, where FIG. 2A: KEGG signaling pathway enrichment barplot of differential genes; FIG. 2B: GSEA (Gene Set Enrichment Analysis) results of genes involved in bile acids metabolism and secretion regulation; FIG. 2C: GSEA results of differential genes by transcriptomics, i.e., results of enrichment of genes involved in bile acids secretion; FIG. 2D: FPKM (Fragments Per Kilo base of exon model per Million mapped fragments) values of the expression of bile acid synthesis and metabolism regulation related genes; when compared with Normal, *P<0.05, **P<0.01, ***P<0.001, ****P<0.0001; and when compared with UC model, ###P<0.001;

FIGS. 3A-3D show the intervention effect of the aqueous extract from Linderae Radix (WY) on TGR5-Src/Yap signaling pathway in the colon with UC, where FIG. 3A: FPKM values of the expression of TGR5-Src/Yap signaling pathway related genes; FIG. 3B, FIG. 3C, and FIG. 3D: expression of TGR5, Src and Yap1 proteins in the colonic tissue of mice in each group, respectively (immunohistochemical DAB staining, 200×), compared with Normal, *P<0.05, **P<0.01;

FIGS. 4A-4G show the regulatory effect of the aqueous extract from Linderae Radix (WY) on the expression of Lgr5 gene and genes involved in ISC activity regulation in the colon of UC mice, where FIG. 4A and FIG. 4B: volcano plots of differential genes by RNA-Seq (NC/Normal-normal control, MC/UC model-UC control; WY-Linderae Radix intervention group); FIG. 4C: FPKM values of ISC stemness markers, FIG. 4D: FPKM values of ISC activity function regulatory genes, FIG. 4E: FPKM values of cell proliferation markers, FIG. 4F: FPKM values of markers on the intestinal cells that have differentiated and matured, and FIG. 4G: LGR5 protein expression in the colonic tissue of mice in each group (immunofluorescence, 200×), when compared with Normal, *P<0.05, **P<0.01, ***P<0.001, ****P<0.0001; and when compared with UC model, #P<0.05, ###P<0.001;

FIGS. 5A-5D show results of the intervention effect of the aqueous extract from Linderae Radix (WY) on the colonic epithelial ultrastructure and the expression of genes involved in mitochondrial function regulation in UC mice, where FIG. 5A: colonic epithelial ultrastructure (transmission electron microscope; 15000×, 30000×); FIG. 5B: heatmap of differential genes by RNA-Seq; FIG. 5C and FIG. 5D: GO enrichment barplot of cellular composition related genes by RNA-Seq;

FIGS. 6A-6F show the effect of the aqueous extract from Linderae Radix (WY) on the expression of genes related to colonic mitochondrial function and dynamics regulation in UC mice, where FIGS. 6A-6C: GSEA results of genes involved in mitochondrial function and dynamics regulation; FIG. 6D: FKPM values of major genes involved in mitochondrial fission and fusion regulation; FIG. 6E and FIG. 6F: expression of Opal protein in the colonic tissue (DAB staining, 200×), when compared with Normal, *P<0.05; and when compared with UC model, #P<0.05.

### DETAILED DESCRIPTION OF THE EMBODIMENTS

The present disclosure provides use of an aqueous extract from Linderae Radix in the preparation of a medicament for preventing and treating ulcerative colitis.

In the present disclosure, there are no particular limitations on the specific source of Linderae Radix, so long as common commercially available products in the art. In the present disclosure, a method for preparing the aqueous extract from Linderae Radix preferably includes the steps of: adding Linderae Radix in water and soaking, reflux extracting the Linderae Radix for 1.0 h-2.0 h at 60° C.-80° C., recovering obtained crude extract under reduced pressure, and concentrating the crude extract to obtain the aqueous extract from Linderae Radix.

In the present disclosure, the water is preferably pure water, and the mass-to-volume ratio of the Linderae Radix to the water is preferably 1 g:10 mL. The time required for the soaking is preferably 30 min-60 min, more preferably 40 min-50 min, and the soaking is preferably carried out at room temperature. The reflux extracting is preferably carried out in water bath, the temperature is preferably 65° C.-80° C., the time required for the reflux extracting is preferably 1.2 h-1.8 h, and the reflux extracting is preferably carried twice. In the present disclosure, the parameters of the second reflux extracting, such as the amount of water added, the reflux extracting temperature and the time required, are the same as those of the first reflux extracting.

In the present disclosure, the aqueous extract from the Linderae Radix preferably plays a role by maintaining the function of goblet cells and restoring intestinal mucus barrier, more preferably plays a role by reducing the damage to colonic epithelial mitochondria and increasing the amount of mitochondria, and further preferably plays an anti-ulcerative colitis role by activating “bile acids-mitochondrial” signaling to enhance Lgr5⁺ ISC activity to further promote the regeneration and repair of intestinal epithelial cells. It is shown that the aqueous extract from the Linderae Radix has a regulatory effect on intestinal stem cells with ulcerative colitis.

In the present disclosure, the ulcerative colitis is a dextran sodium sulfate-induced ulcerative colitis. Although both TNBS (2,4,6-trinitrobenzenesulfonic acid) and dextran sodium sulfate (DSS) may cause ulcerative colitis, they each have their characteristics and different modes of action. Specifically, TNBS triggers the pathological changes of ulcerative colitis mainly by inducing immune responses. When TNBS is injected directly into the colon of an animal, it triggers an abnormal response in the immune system, resulting in damage to the colonic mucosa and an inflammatory response. TNBS modeling often results in significant ulcer formation and immune responses, characterized by intestinal wall thickening, necrosis, ulcer formation, local inflammatory cell infiltration, etc. While DSS may induce damage to intestinal mucosal epithelial cells, causing ulcerative colitis. DSS modeling does not involve activation of the immune system, but directly leads to impaired mucosal barrier function, inflammatory responses and ulcer formation. Due to the ability to rapidly induce colonic inflammation and short experimental period, DSS model is commonly used for preliminary evaluation on inflammatory response and treatment, which is characterized by mucosal damage, ulcer formation, inflammatory cells infiltration, etc. Therefore, the TNBS-induced ulcerative colitis is different from the DSS-induced ulcerative colitis. The present disclosure focuses on the aqueous extract from Linderae Radix having the effect of preventing and treating ulcerative colitis caused by DSS-mediated intestinal mucosal barrier damage.

The present disclosure also provides a medicament for preventing and treating ulcerative colitis, where the medicament contains the aqueous extract from Linderae Radix as the sole active ingredient. In the present disclosure, the dosage form of the medicament preferably includes an aqueous solution for injection, a powder-injection, a pill, a powder, a tablet, a patch, a suppository, an emulsion, a cream, a gel, a granule, a capsule, an aerosol, a spray, an inhalation powder, a sustained release agent and a controlled release agent. In the present disclosure, the medicament further includes a pharmaceutically acceptable auxiliary material, the pharmaceutically acceptable auxiliary material may be conventionally used in various preparations, such as but not limited to, an isotonic agent, a buffer, a flavoring agent, an excipient, a filler, a binder, a disintegrant, a lubricant, etc.; the pharmaceutically acceptable auxiliary material may also be selected for adaptation to the active substance, such as an emulsifier, a solubilizer, a bacteriostat, an analgesic, an antioxidant, etc. Such auxiliary materials can effectively improve the stability and solubility of the active ingredient or change the release rate, absorption rate, etc. of the active ingredient to improve the metabolism of the active ingredient in an organism, thereby enhancing the administration effect of the active ingredient. In addition, the pharmaceutically acceptable auxiliary material, may also be such as, but not limited to, gelatin, albumin, chitosan, polyether and polyester polymer materials (such as, but not limited to, polyethylene glycol, polyurethane, polycarbonate, and copolymers thereof), that is used to achieve a particular purpose or mode of administration, such as sustained-release administration, controlled-release administration and pulsed administration. The effects of the auxiliary material recited in the present disclosure include, but are not limited to, improving therapeutic effect, improving bioavailability, reducing toxic and side effects, improving patient compliance, etc.

The technical solutions provided by the present disclosure will be described in detail in conjunction with the examples below, which cannot be construed as limiting the scope of protection of the present disclosure.

Methods in the following examples are all conventional methods, unless otherwise specified.

The materials, reagents, etc. used in the following examples are all commercially available, unless otherwise specified.

Experimental materials used in the following examples:

Animals: Thirty-six 6-week-old SPF (Specific Pathogen Free) male C57 mice (weighing 20±2 g) were provided by Zhejiang Chinese Medical University Laboratory Animal Research Center, with laboratory animal certification of fitness number: 20221019Abzz0100999503; license number: SCXK (Zhejiang) 2022-0005. At a temperature of (23±2)° C., relative humidity of 40%-60%, the mice were fed in the lab with regular chow, and had free access to water. All experiments were approved by the Animal Care and Use Committee of the Zhejiang Chinese Medical University Laboratory Animal Research Center. All procedures involving in mouse studies were in accordance with the ethical principles of animal welfare. All surgical procedures were designed to minimize animal suffering and reduce the number of animals used.

Drugs and reagents: Linderae Radix decoction pieces were provided by Zhejiang Chinese Medical University Chinese Medicine Yin pian Co., Ltd. (Hangzhou, China); sulfasalazine (SASP) (Cat. No.: H19994081; purchased from Shanghai Sine Tianping Pharmaceutical Co., Ltd.); dextran sulfate sodium (DSS; 36,000-50,000 kDa; Cat. No.: 116570400 50 preps; MP Biomedicals, USA), and all other reagents being commercially available.

Statistical analysis of data in the following examples:

Statistical analysis was performed on data using GraphPad Prism 9.0 software. The data were shown as mean±SD, comparisons between multiple groups using one-way analysis of variance (ANOVA) and multiple comparisons were performed, and P value<0.05 was considered as statistically significant.

### Example 1

### Preparation of the Aqueous Extract From Linderae Radix

40 g of Linderae Radix decoction pieces were taken, 400 mL of pure water was added according to a material-liquid ratio of 1 g:10 mL, soaking was performed for 45 min, heating was performed in a water bath at 80° C., reflux extracting for 1 h, and the filtrate was taken for later use; 400 mL of pure water was added to the remaining raw material according to a material-liquid ratio of 1 g:10 mL, heating was performed in a water bath at 80° C., reflux extracting for 1 h, and the filtrate was taken for later use; and the two filtrates were combined, and the resulting crude extract was recovered under reduced pressure and concentrated to 200 mL (0.2 g crude drug/mL) to obtain the aqueous extract from Linderae Radix.

### Example 2

The aqueous extract from Linderae Radix obtained in Example 1 was taken and administered at an administration dose of 2 g/kg to the laboratory animals, recorded as a high dose Linderae Radix group.

50 mL of the aqueous extract from Linderae Radix (0.2 g crude drug/mL) obtained in Example 1 was taken, 50 mL of pure water for experiment was added to dilute to a concentration of 0.1 g crude drug (Linderae Radix)/mL, and the resulting aqueous extract from Linderae Radix was administered at an administration dose of 1 g/kg to the laboratory animals, recorded as a medium dose Linderae Radix group.

30 mL of the aqueous extract from Linderae Radix (0.2 g crude drug/mL) obtained in Example 1 was taken, 90 mL of pure water for experiment was added to dilute to a concentration of 0.05 g crude drug (Linderae Radix)/mL, and the resulting aqueous extract from Linderae Radix was administered at an administration dose of 0.5 g/kg to the laboratory animals, recorded as a low dose Linderae Radix group.

Nine tablets of sulfasalazine (SASP, 0.25 g×60 tablets/box) were taken into a glass mortar and ground into powder, the powder was sieved, the sugar coat residue was removed, then 50 mL of pure water was added to prepare a suspension (0.045 g crude drug/mL) (administration dose: 0.45 g/kg), and the suspension was placed in a refrigerator at 4° C. for later use.

The thirty-six animals underwent one-week acclimation prior to the start of the experiment. According to the body weight, the animals were randomly divided into a normal group (Normal), a model group (Model), a positive drug group (SASP, 0.045 g/kg), a high dose Linderae Radix group (2 g/kg), a medium dose Linderae Radix group (1.0 g/kg), and a low dose Linderae Radix group (0.5 g/kg), with 6 animals in each group. The mice in each group except for those in the normal group were free to drink 2.5% DSS solution for 9 consecutive days to establish ulcerative colitis (UC) models. If the model mice experienced phenomena such as weight loss, mental sluggishness, loss of appetite, loose and viscous stool, anal fissure and rectal prolapse, the modeling was successful, and the number of days was increased if the modeling criteria were not met until the modeling was successful. Simultaneously with modeling, the mice in the normal group were given an equal volume of distilled water, and the mice in the remaining groups were given corresponding drug via gavage at 0.1 mL/10 g body weight for 9 consecutive days.

At the end of the whole experimental period, the mice were fasted overnight, whole blood was collected from the orbit, and the serum was isolated for subsequent detection. The mice were anesthetized and dissected, the rectal segment from 1-2 cm away from the anus to the end of the cecum (colonic segments) were taken from the mice, respectively, the mesentery was cleaned, the colonic mucosa was visually observed for lesions, the length of the colonic tissue of the mice was measured, and the colonic segments were frozen at −80° C. for subsequent analysis. A part of the colonic segment was fixed in 10% formalin and embedded in paraffin for hematoxylin and eosin (H&E) staining to evaluate the extent of lesion in the colonic tissue.

During the experiment, the mice in each group were observed regularly every day for eating, drinking, hair color, activity and stool character, weighed, and observed for occult blood in stool, and the disease activity index (DAI) was scored (see Table 1). The scoring criteria for occult blood in stool are as shown in Table 2.

TABLE 1
Scoring criteria for DAI

| | Weight | | |
|---|---|---|---|
| Score | loss (%) | Stool character | Hematochezia |
| 0 | None | Normal stool | Occult blood (−) |
| 1 | 1-5 | Between normal stool and | Occult blood (+) |
| | | semi-loose stool | |
| 2 | 6-10 | Semi-loose stool | Occult blood (++) |
| 3 | 11-15 | Between semi-loose stool | Occult blood (+++) |
| | | and loose stool | |
| 4 | >15 | Loose stool | Gross blood stool |

Note:
The percent weight loss per day for animals was calculated as follows: Weight loss (%) = (body weight at a certain time point after modeling − body weight before modeling)/body weight before modeling × 100%. Normal stool is dry and small well-shaped ball, semi-loose stool is pasty or semi-shaped but does not stick to the anus, and loose stool is liquid and sticks to the anus.

TABLE 2
Scoring criteria for occult blood in stool

| O-benzidine titration | Stool/occult blood | Score |
|---|---|---|
| No blue or green color developed after | Occult blood (−) | 0 |
| three minutes | | |
| Develop a light blue color within 1 to 3 | Occult blood (+) | 1 |
| minutes | | |
| Develop a blue color gradually within | Occult blood (++) | 2 |
| 0 to 60 seconds | | |
| Develop a blue color immediately | Occult blood (+++) | 3 |
| Develop a dark blue color immediately | Watery stool (gross | 4 |
| (gross blood stool) | blood stool) | |

Observation of the pathological change of colonic tissue by hematoxylin-eosin (H&E) staining and alcian blue/periodic acid-Schiff (AB-PAS) staining:

1.0 cm of colon was fixed in 10% formalin, dehydrated with ethanol with gradient concentration, permeabilized with xylene, embedded in paraffin, and cut into 4 μm sections for H&E staining and AB-PAS staining.

Observation of the ultrastructural change in colonic tissue by transmission electron microscopy:

About 1 mm³ of colonic tissue was taken, sequentially fixed in 2.5% glutaraldehyde and 1% osmic acid, dehydrated with ethanol in gradient concentration and acetone solutions, embedded with different ratios of acetone+embedding solution, cured at gradient temperature in an oven, and cut into ultrathin sections, the section was double-stained with 3% uranium acetate-lead citrate, and the morphologies of colonic epithelial villi, epithelial intercellular space, columnar cell structure and organelles such as mitochondria were observed by transmission electron microscopy.

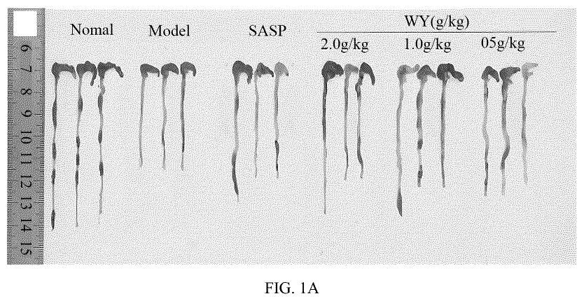

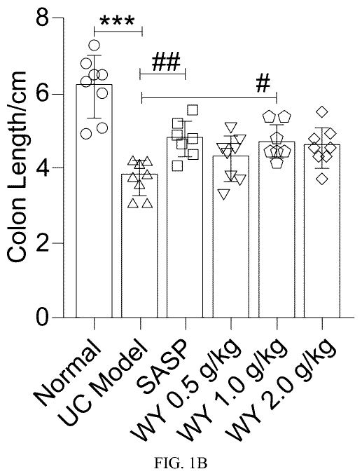

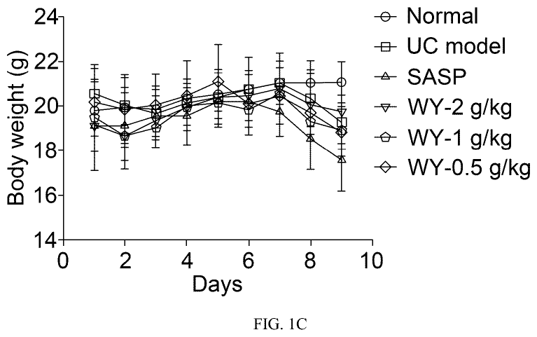

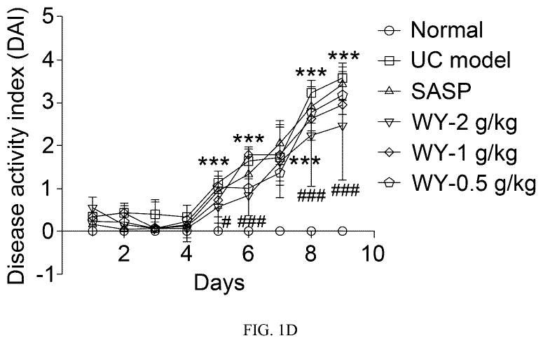

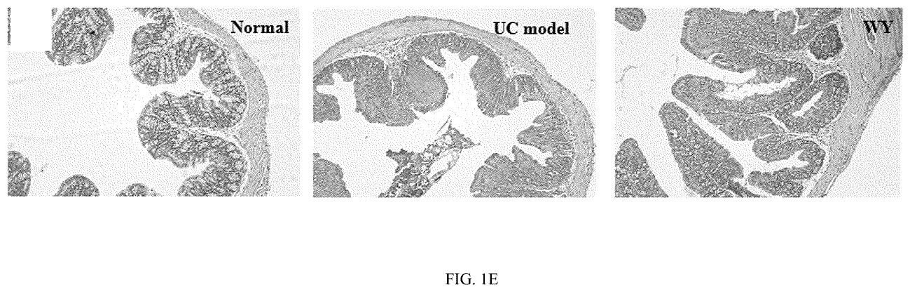

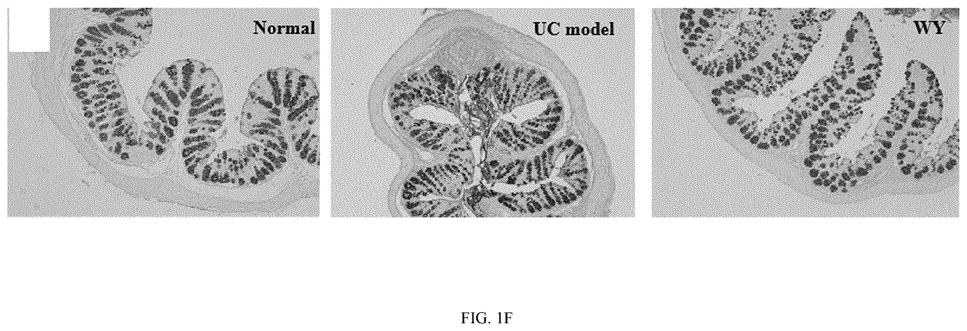

The results are as shown in FIGS. 1A-1F, and it can be seen that compared with the mice in the normal control group, the UC mice have significantly shortened colon (P<0.001) and significantly increased colon disease activity index (DAI) (P<0.001), and that each dose of the aqueous extract from Linderae Radix can effectively improve disease symptoms such as weight loss and hematochezia, increase colorectal length (P<0.01) and decrease DAI (P<0.05) in UC mice (see FIGS. 1A-1D). The results of HE staining show that in the colon of UC mice, multiple ulcer lesions are visible, and there is a significant damage to the architecture of colonic crypts (see FIG. 1E); the high dose Linderae Radix (WY group) can effectively reduce the amount and area of colonic epithelial ulcer lesions, and the results of AB-PAS staining (see FIG. 1F) show that compared with the mice in the normal control group, the mice in the UC model group have significant decrease in amount of goblet cells and mucus secretion and have severely atrophied glandular cells in the colon; and the aqueous extract from Linderae Radix can reduce DSS-induced goblet cell depletion and maintain goblet cell mucus secretion compared with that in the model control group. These results indicate that the aqueous extract from Linderae Radix can improve the pathological symptoms of DSS-induced colitis.

Immunohistochemical detection of intracellular localization of TGR5, Src, YAP1 and OPA1 expression in colonic tissue:

Colonic tissue samples were fixed in 10% formalin solution, embedded in paraffin, sectioned, and subjected to conventional IHC to detect the expression of TGR5 (antibody for detection: ab72608, ABCAM, USA; 1:100), Src (antibody for detection: ET1702-03, HuaBio, China; 1:50-200), YAP1 (antibody for detection: 13584-1-AP, Proteintech, China; 1:50-500) and OPA1 (antibody for detection: ET1705-9, HuaBio, China; 1:50-200) in colonic epithelium. Each slide was observed with a light microscope and images in three random fields of view were captured. The expression of TGR5, Src, YAP1 and OPA1 were analyzed semi-quantitatively with IOD by Image J software (National Institutes of Health, Bethesda, USA). The paraffin slides of colonic tissue were deparaffinized in xylene and hydrated in different concentrations of ethanol, antigen retrieval was performed in a citrate buffer, endogenous peroxidase was blocked with 3% H₂O₂, and primary antibody src was added dropwise and incubated overnight at 4° C. Washing was performed with PBS, HRP-labeled goat anti-rabbit secondary antibody was added dropwise and incubated for 30 min at 37° C., a freshly prepared DAB color development solution was added dropwise, color development was allowed, and positive expression was observed microscopically.

Immunofluorescence detection of expression of Lgr5 protein in colon:

Colonic tissue was mounted with paraffin and cut into sections with a thickness of 4 μm. The sectioned sample was deparaffinized in xylene, and rehydrated with ethanol of gradient concentration, and antigen retrieval was performed. First, dilution was performed using QuickBlock immunostaining blocking buffer (P0260, Beyotime, China), and blocking treatment was performed on the tissue for 10 min. Subsequently, rabbit-derived Lgr5 antibody (bs-20746R, Bioss, China; 1:100-500) was added to the sample, and incubated overnight at 4° C. Then washing was performed with PBS three times to remove excess primary antibody. The sections were then incubated with corresponding secondary antibody (bs-0296G-FITC, Bioss, China, Goat Anti-Mouse IgG H&L antibody) for 30 min at 37° C. in the dark. Finally, the sections were blocked using DAPI solution and observed after 10 min using a Zeiss positive fluorescence microscope (AXIO SCOPE.A1, Carl Zeiss AG, Germany).

Change in expression of bile acids metabolism related genes Cyp27 alpha1 and Baat, TGR5-Src/Yap signalling pathway related genes TGR5, GRK2, beta-Arrestin, Src, Yes1 and Yep1, stem cell proliferation and differentiation related genes Hmgcs2, PCNA, KRT20, Villlin, MUC2, Lgr5, Hopx and ASCL2, and mitochondrial dynamics related genes Drp1 (DNMIL), Fis1, Mfn1 and Opa1 in colonic tissue was analyzed by RNA sequencing (RNA-Seq, transcriptome sequencing):

Transcriptome sequencing was performed for colonic tissues in each group (normal group, model group, and high dose Linderae Radix group), and 2×150 bp paired-end sequencing (PE150) was performed on Illumina Novaseq™ 6000 (LC-Bio Technologies (Hangzhou) Co., Ltd., Hangzhou, China). Differentially expressed genes (DEGs) were selected using the R package on the basis of comprehensive consideration of the fold change and p value of each gene [p value<0.05, absolute value log2 (fold change)>=1]. Sequencing data were analyzed on the OmicSmart platform (www.omicsmart.com). After acquiring the sequencing data, the off-line data were first filtered to acquire high quality sequencing data, and the high quality sequencing data were aligned with the reference genome of the project species for gene expression quantification, GSEA, gene difference analysis, enrichment analysis, etc.

The results of the above transcriptomic assay (RNA sequencing) were as follows:

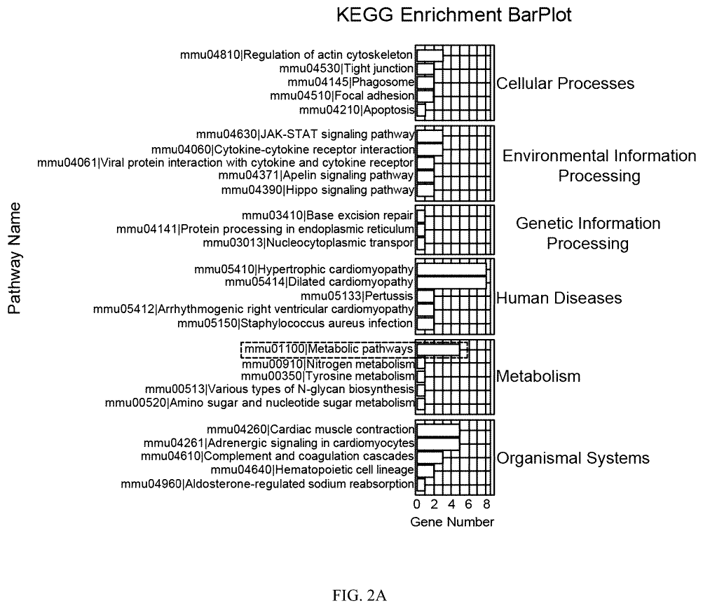

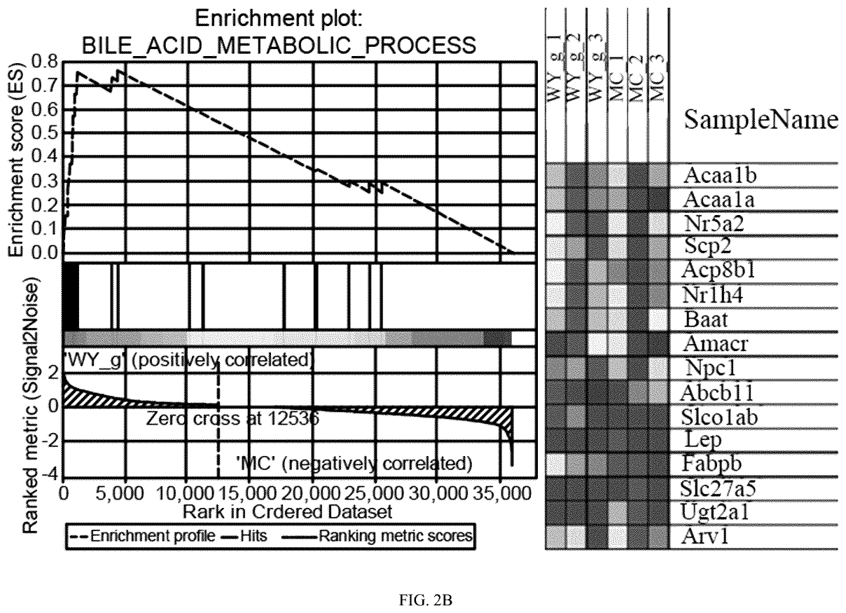

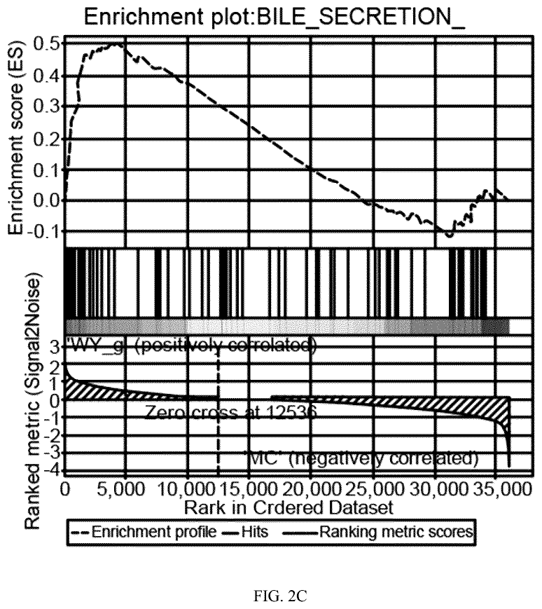

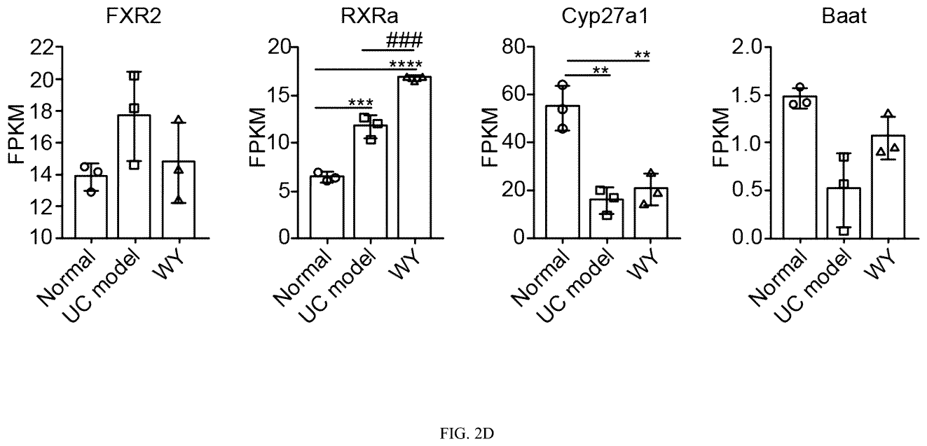

Regulatory effect of the aqueous extract from Linderae Radix on bile acids in pathological states: the effect of the aqueous extract from Linderae Radix (WY) at a high dose (2 g/kg) on bile acids metabolism in DSS-induced UC mice was analyzed by transcriptomics. It was found that in UC mice, the genes involved in bile acids metabolism processes regulation are significantly enriched and the bile secretion signaling pathway is significantly affected, indicating that the intervention of the aqueous extract from Linderae Radix can upregulate the expression of genes Cyp27alpha 1 and Baat involving in the bile acids synthesis and transportation regulation (P<0.05) (see FIGS. 2A-2D).

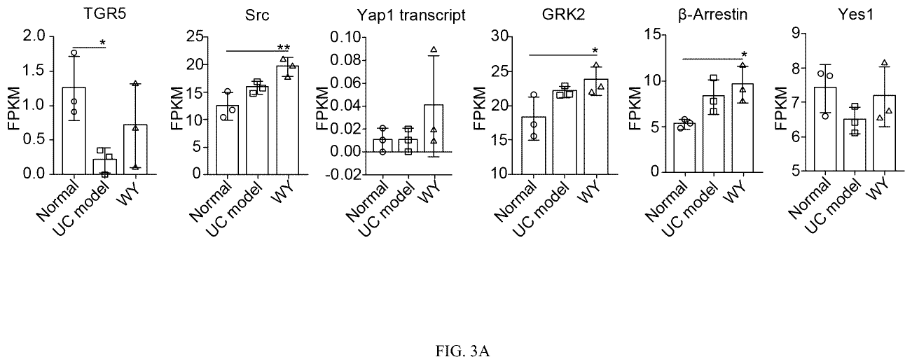

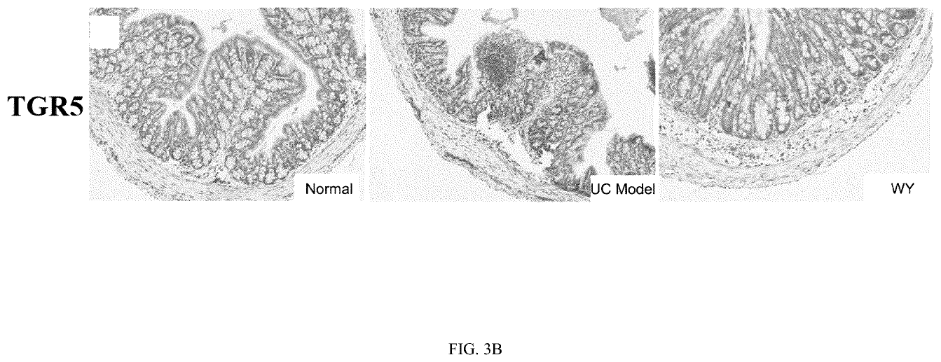

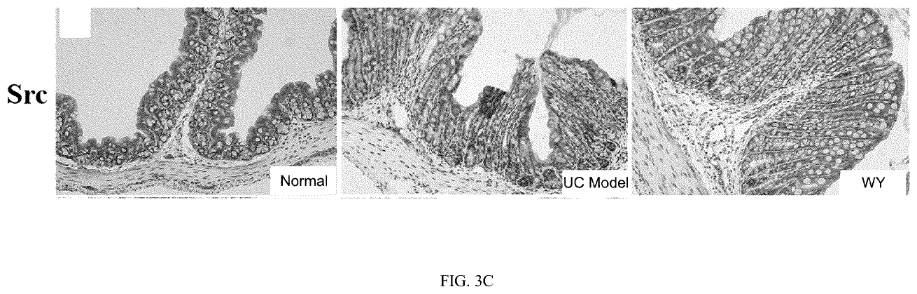

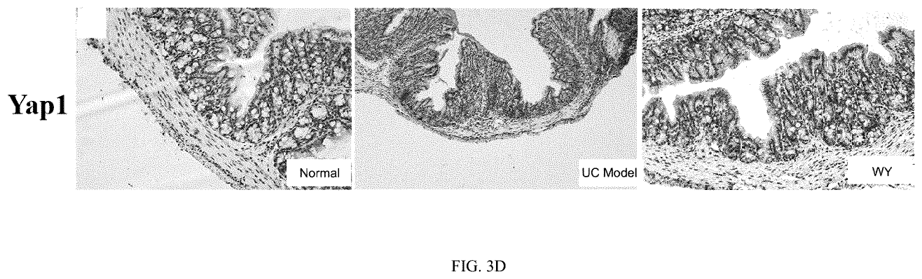

Intervention effect of the aqueous extract from Linderae Radix on bile acids-TGR5 signaling: the regulatory effect of the aqueous extract from Linderae Radix (WY) at a high dose (2 g/kg) on the TGR5-Src/Yap signaling pathway in the colonic tissue of DSS-induced UC mice was observed by transcriptomics. It was found that the gene expression of TGR5 in UC mice was significantly down-regulated compared with that in the normal control group (P<0.05) (see FIG. 3A), indicating that the intervention of the aqueous extract from Linderae Radix can upregulate the expression of genes TGR5, GRK2 (P<0.05), beta-Arrestin (P<0.05), Src (P<0.05), Yes1, Yep1, etc. involving in TGR5-Src/Yap signalling pathway (see FIG. 3A). The immunohistochemical results also show that the aqueous extract from Linderae Radix (at a high dose of 2 g/kg) can increase the expression of TGR5, Src and Yep1 proteins in the colonic epithelium (see FIG. 3B-FIG. 3D).

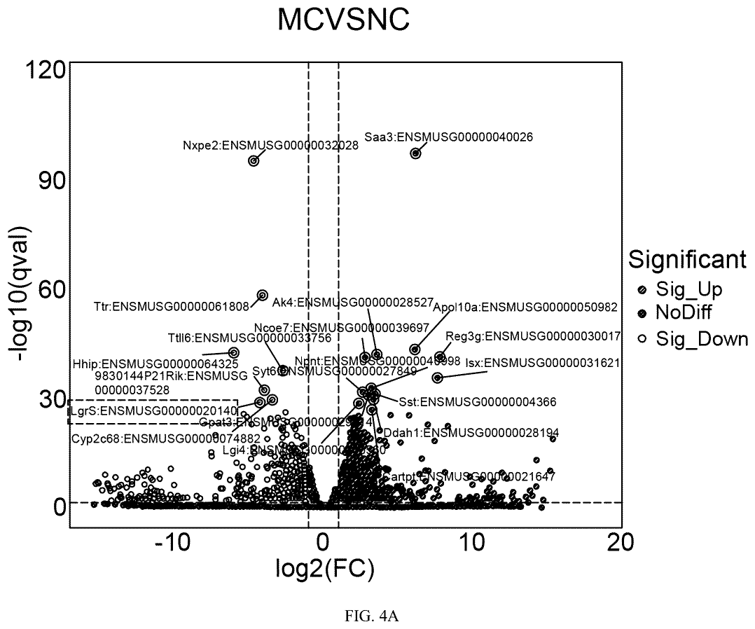

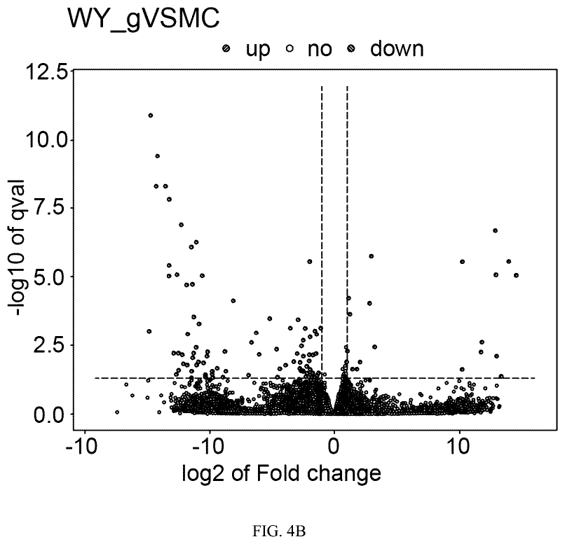

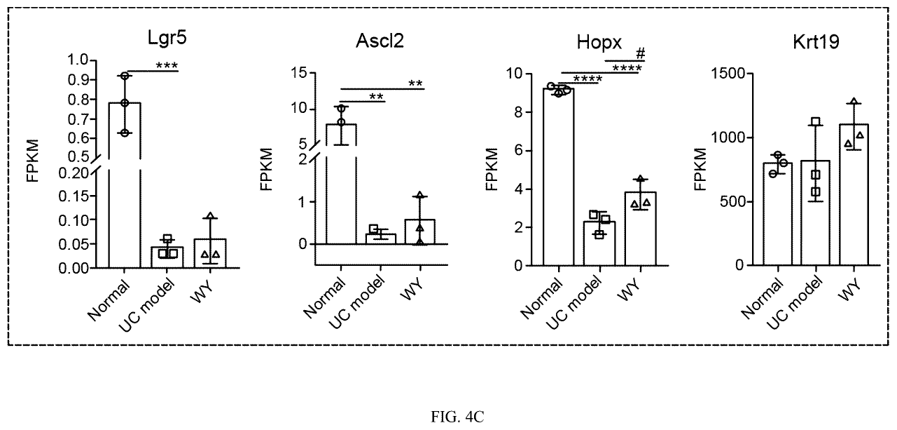

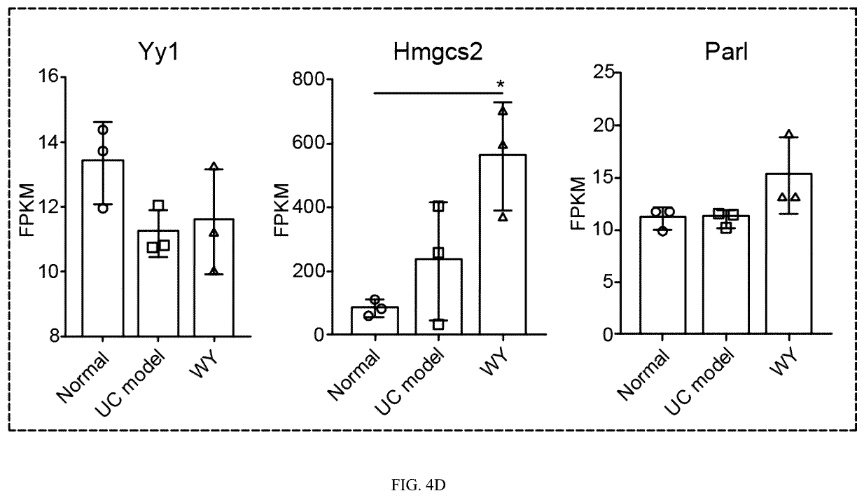

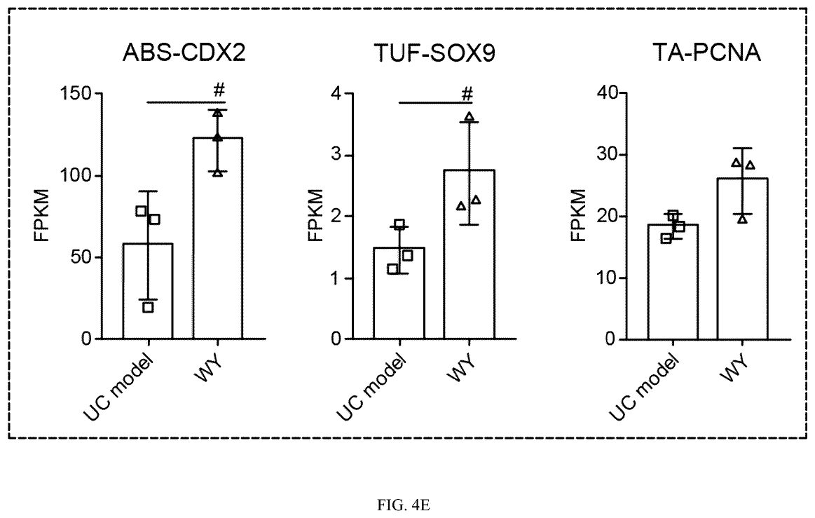

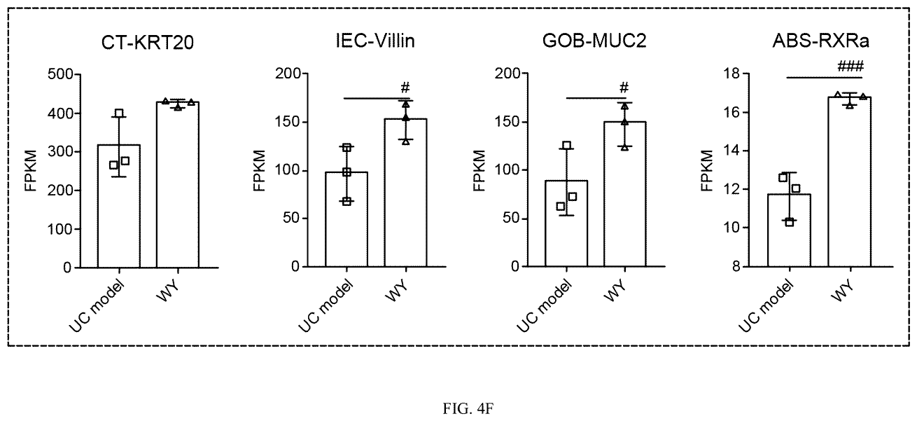

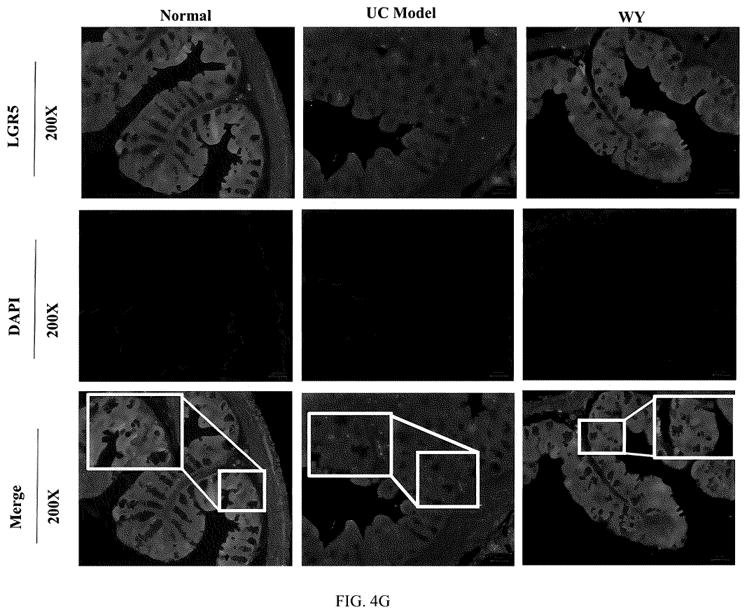

Intervention effect of the aqueous extract from Linderae Radix on Lgr5 expression and genes involving in Lgr5⁺ ISC proliferation and differentiation regulation in colon of UC mice: the regulatory effect of the aqueous extract from Linderae Radix (WY) at a high dose (2 g/kg) on Lgr5⁺ ISC activity was preliminarily observed by transcriptomic studies (see FIG. 4A). It was found that the gene expression of Lgr5 in the colon of UC mice was significantly down-regulated (P<0.001), indicating that the intervention of the aqueous extract from Linderae Radix can up-regulate the expression levels of intestinal cell stemness related gene Lgr5, Hopx and Lgr5 specific transcript ASCL2 (P<0.01) (see FIG. 4C); meanwhile, the immunofluorescence results show that the immunofluorescence signal of Lgr5 in UC mice is weak, but the expression of Lgr5 in UC mice is significantly increased after receiving the treatment with Linderae Radix (see FIG. 4G). In addition, the expression levels of genes that support Lgr5⁺ ISC activity and function, such as Hmgcs2, of cell proliferation markers and various maturation and differentiation markers of intestinal cells, such as PCNA, KRT20, Villlin and MUC2, were also higher than those in UC model mice (see FIG. 4D-FIG. 4F).

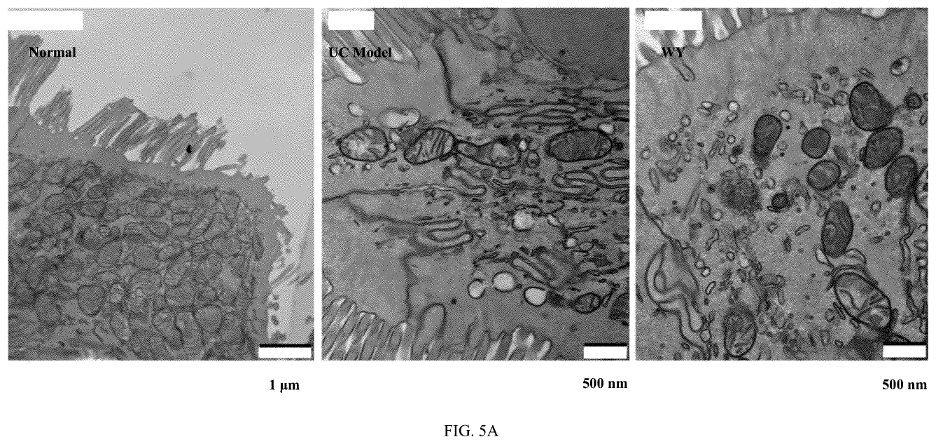

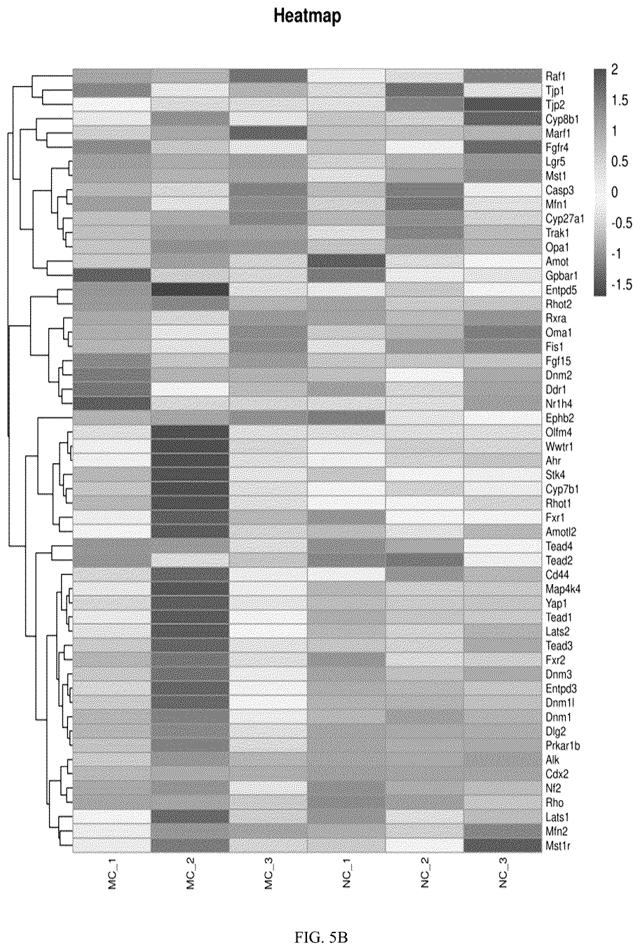

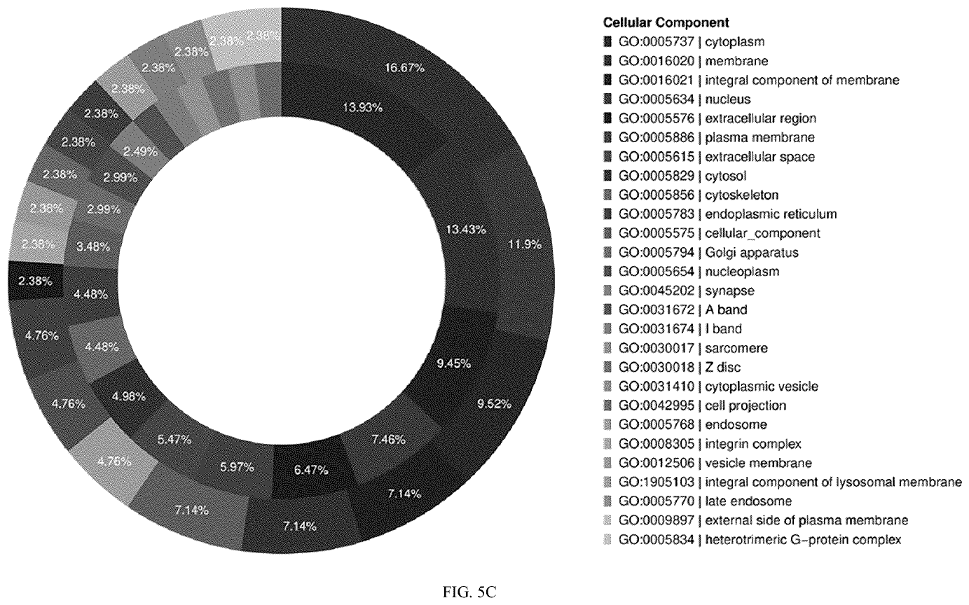

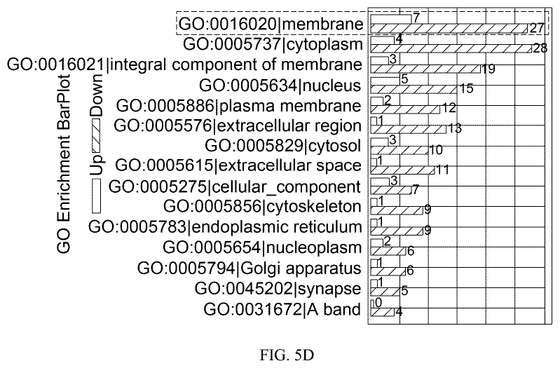

Intervention effect of the aqueous extract from Linderae Radix on genes involved in morphology and function regulation of mitochondria in colonic epithelial: the regulatory effect of the aqueous extract from Linderae Radix (WY) at a high dose (2 g/kg) on the morphology and function of colonic epithelial mitochondria in UC mice was observed by transmission electron microscopy and transcriptomics. The results by transmission electron microscopy show that compared with normal mice, the UC mice have the following characteristics in the colonic epithelial cells: the amount of mitochondria is significantly reduced, the crista structure is altered or disappeared, and the cells are atrophic; and the colonic cells of UC mice in the Linderae Radix intervention group have a larger amount of larger-volume mitochondria and relatively intact crista structure (see FIG. 5A). The results by transcriptomic analysis show that mitochondrial dynamics and function regulatory genes are differentially expressed and that mitochondrial membrane regulatory genes are significantly enriched (see FIG. 5B-FIG. 5D).

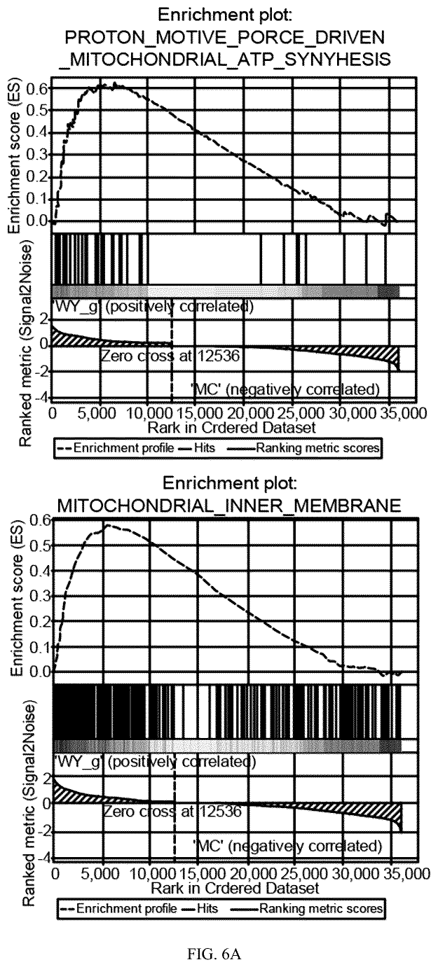

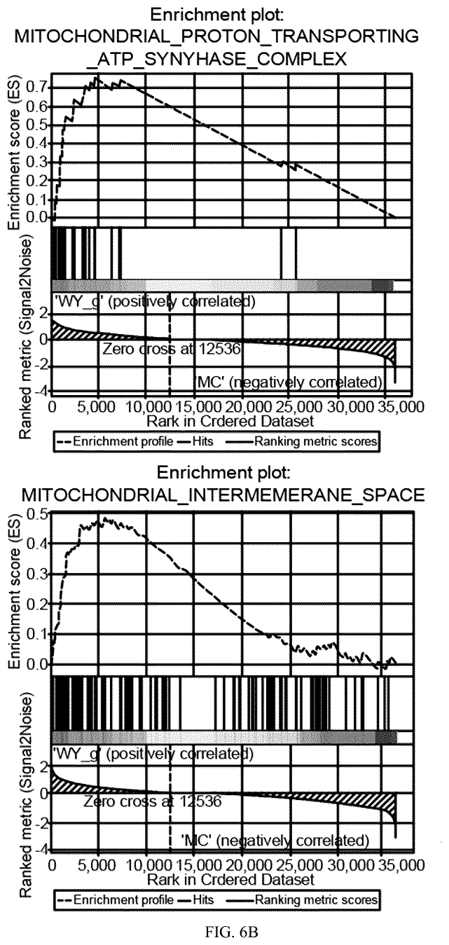

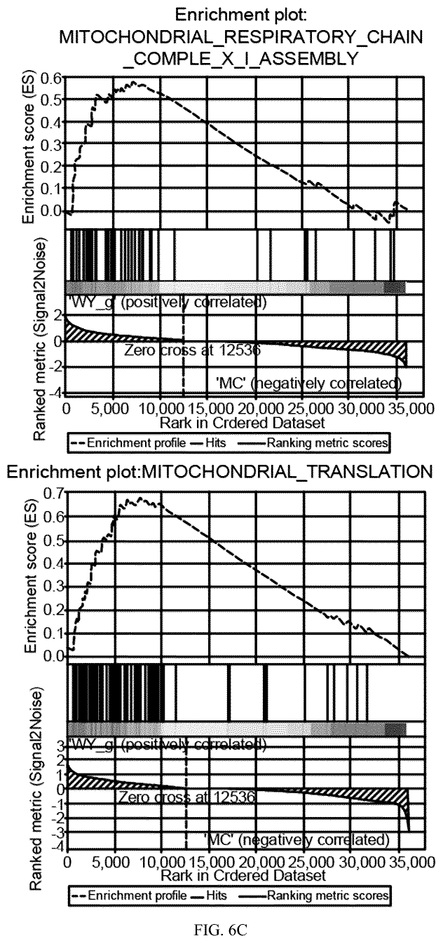

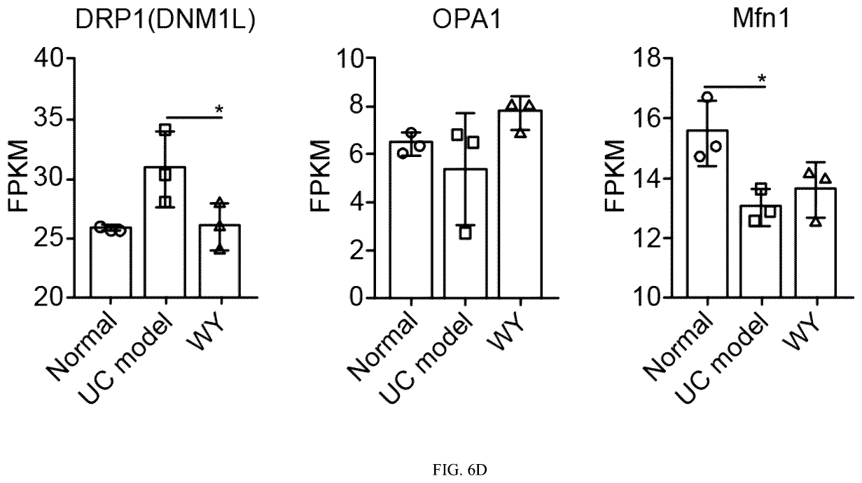

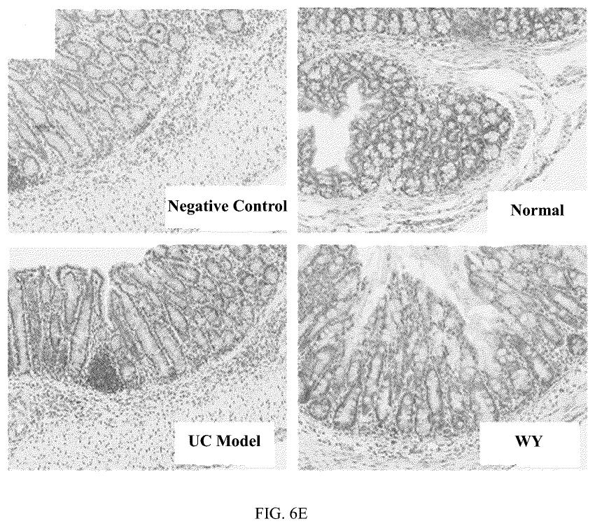

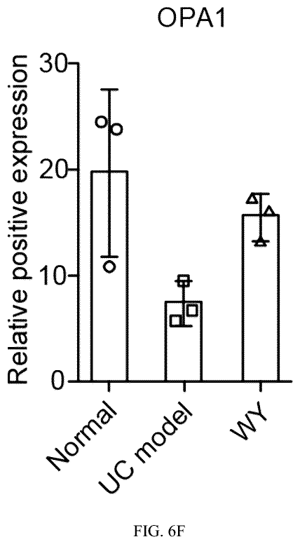

To further observe the effect of the aqueous extract from Linderae Radix at a high dose (2 g/kg) on the colonic mitochondrial dynamics in UC mice, the expression of genes involving in mitochondrial fusion and fission regulation was analyzed. The results by transcriptome sequencing analysis show abnormal expression of genes related to mitochondrial fission and fusion dynamics regulation and significant enrichment of genes involving in the regulation to space in mitochondrial inner membrane and between the inner and outer mitochondrial membrane in UC mice (see FIGS. 6A-6C), and show that the expression of genes Drp1 (DNMIL) (P<0.05) and Fis1 involved in regulation of mitochondrial fission are significantly up-regulated, and that the expression of fusion genes Mfn1 (P<0.05) and Opal are significantly down-regulated, indicating that the aqueous extract from Linderae Radix can reversely regulate the abnormal expression of the above genes, and positively regulate the expression of genes related to mitochondrial function regulation, such as regulation to mitochondrial ATP synthesis complex and respiratory chain complex 1 assembly (see FIG. 6D-FIG. 6F).

The descriptions above are merely the preferred embodiments of the present disclosure. It should be noted that several improvements and modifications may also be made by the ordinary artisans concerned without departing from the principle of the disclosure, and these improvements and modifications should also be considered within the protection scope of the disclosure.

What is claimed is:

1. A method for preparing a medicament for preventing and treating ulcerative colitis, comprising
using an aqueous extract from Linderae Radix.

2. The method of claim 1, wherein a method for preparing the aqueous extract from Linderae Radix comprises:
adding the Linderae Radix into water and soaking,
reflux extracting the Linderae Radix for 1.0 h-2.0 h at 60° C.-80° C.,
recovering obtained crude extract under reduced pressure, and
concentrating the crude extract to obtain the aqueous extract from Linderae Radix.

3. The method of claim 2, wherein a mass-to-volume ratio of the Linderae Radix to the water is 1 g:10 mL.

4. The method of claim 2, wherein a time required for the soaking is 30 min-60 min.

5. The method of claim 2, wherein the reflux extracting is carried out twice.

6. The method of claim 1, wherein the aqueous extract from Linderae Radix plays a role by maintaining function of goblet cells and restoring intestinal mucus barrier.

7. The method of claim 1, wherein the aqueous extract from Linderae Radix plays a role by reducing damage to colonic epithelial mitochondria and increasing an amount of mitochondria.

8. The method of claim 1, wherein the aqueous extract from Linderae Radix plays an anti-ulcerative colitis role by activating “bile acids-mitochondrial” signaling to enhance Lgr5⁺ ISC activity to further promote regeneration and repair of intestinal epithelial cells.

9. The method of claim 1, wherein the ulcerative colitis is a dextran sodium sulfate-induced ulcerative colitis.

10. A medicament for preventing and treating ulcerative colitis, wherein the medicament contains the aqueous extract from Linderae Radix in the method of claim 2 as a sole active ingredient.

11. The medicament of claim 10, wherein a mass-to-volume ratio of the Linderae Radix to the water is 1 g:10 mL.

12. The medicament of claim 10, wherein a time required for the soaking is 30 min-60 min.

13. The medicament of claim 10, wherein the reflux extracting is carried out twice.
